(19) United States
(12) Patent Application Publication
(10) Pub. No.: US 2023/0114154 A1
(43) Pub. Date: Apr. 13, 2023

(54) COATED SPONGES

(71) Applicants: Dow Global Technologies LLC, Midland, MI (US); Dow Silicones Corporation, Auburn, MI (US)

(72) Inventors: Yasmin N. Srivastava, Sugar Land, TX (US); Cody Schoener, Midland, MI (US); Yihan Liu, Midland, MI (US)

(21) Appl. No.: 17/908,035

(22) PCT Filed: Jan. 27, 2021
(86) PCT No.: PCT/US2021/015200

§ 371 Date: Aug. 30, 2022

Related U.S. Application Data
(60) Provisional application No. 63/007,453, filed on Apr. 9, 2020.

(51) Int. Cl. A61K 8/02; A61K 8/895; A61K 8/892; A61K 8/11; A61K 8/81; A61K 8/31; A61Q 1/02; C08L 91/06; A45D 34/04

(52) U.S. Cl. CPC A61K 8/0204; A45D 34/04; A61K 8/11; A61K 8/31; A61K 8/892; A61K 8/895; A61K 8/8152; A61Q 1/02; C08L 91/06; A45D 2200/1036; A61K 2800/87

(57) ABSTRACT

Sponges for cosmetic and/or medicinal applications are coated with a surface coating. The surface coating contains an elastomeric, non-silicone polymer, a certain silicone polymer, an encapsulated phase change material and optionally ceramic particles. The coated sponges have desirable haptic properties and efficiently absorb and release make-up and medicines for application to the skin.

This invention relates to sponges that are useful in cosmetic applications, in particular for applying and/or removing cosmetic compositions to the skin.

Sponges are commonly used to apply and/or remove cosmetics from or otherwise clean human skin, particularly the face and hands. The sponges may be natural types but more usually are synthetic polymer foams such as polyurethane or silicone polymer foams. The sponges are designed to be soft to enhance haptic properties and avoid skin irritation, and hydrophilic so they absorb make-up and other cosmetics.

Haptic properties are very important to consumers, so methods by which desirable haptic properties can be imparted to sponges are of great interest. Such methods must not unduly interfere with the ability of the sponges to perform their underlying function, which is to apply and/or remove cosmetics from and/or to clean the skin.

Various approaches have been proposed to impart a cooling sensation to wipes, tissues and other facial products. Among those approaches are those described in U.S. Pat. Nos. 8,039,011, 8,894,814 and 9,545,365 and U.S. Published Pat. Application 2014-0242127. These describe lotions to be applied to a tissue or the skin. The lotions contain phase change materials such as low-melting waxes and other low melting polymers. U.S. Pat. No. 9,234,509 describes molded articles coated with a polymeric phase-change material that bonds to the molded substrate via covalent bonding and entanglement mechanisms.

U.S. Published Pat. Application No. 2016-223269 describes polymer films that include a polymeric phase change material.

WO 2017/210439 describes a polyurethane foam having a surface coating that contains an encapsulated phase change material. The foam is used as a bedding material, i.e., for mattresses, pillows and similar items. The surface coating imparts a “cool touch” feature that is a desired point-of-sale feature of those products.

This invention is an article comprising a natural or synthetic sponge and a cured coating of a solid, water-insoluble, elastomeric non-silicone polymer adhered to at least one surface of the sponge, the cured coating further containing (i) at least one silicone polymer having a kinematic viscosity of at least 1 million mm²/s at 25° C. and/or a Williams plasticity number of at least 30 as measured according to ASTM 926; (ii) particles of an encapsulated phase change material, the phase change material having a melting or glass transition temperature of 25 to 37° C. and optionally (iii) ceramic particles having a particle size of up to 50 µm, the silicone polymer constituting 2.5 to 30 weight percent of the combined weight of the non-silicone polymer, silicone polymer, encapsulated phase change material particles and ceramic particles, the encapsulated phase change material constituting 10 to 70 weight percent of the combined weight of the non-silicone polymer, silicone polymer, encapsulated phase change material particles and ceramic particles, and the ceramic particles when present constituting up to 25 weight percent of the combined weight of the non-silicone polymer, silicone polymer, encapsulated phase change material particles and ceramic particles.

The coated sponge is particularly useful for cosmetic applications such as applying and/or removing a cosmetic from the skin. The coated sponge has haptic properties that are quite desirable in such applications and effectively transfers cosmetics to the skin. Similarly, the coated sponge is useful for medical applications that involve applying a medication to the skin and/or removing a foreign substance from the skin. The coated sponge is suitable for cleaning the skin.

The invention in another aspect is a coating composition useful for producing the foregoing article. The coating composition comprises a liquid phase containing water and/or one or more other compounds that are liquid at 23° C. and have a boiling temperature at standard pressure of 40 to 100° C., a water-insoluble elastomeric, non-silicone polymer dispersed in the liquid phase in the form of particles or droplets, at least one silicone polymer having a viscosity of at least 1 million mm²/s at 25° C. and/or a Williams plasticity number of at least 30 as measured according to ASTM 926 dispersed in the liquid phase, particles of an encapsulated phase change material dispersed in the liquid phase, and optionally ceramic particles dispersed in the liquid phase, wherein the silicone polymer constitutes 2.5 to 30 weight percent of the combined weight of the non-silicone polymer, silicone polymer, encapsulated phase change material particles and ceramic particles, the encapsulated phase change material constitutes 10 to 70 weight percent of the combined weight of the non-silicone polymer, silicone polymer, encapsulated phase change material particles and ceramic particles, and the ceramic particles when present constitute up to 25 weight percent of the combined weight of the non-silicone polymer, silicone polymer, encapsulated phase change material particles and ceramic particles.

The invention is also a method of making the coated sponge of the invention, the method comprising applying the foregoing coating composition to at least one surface of a natural or synthetic sponge to form a continuous or discontinuous film thereupon, and curing the coating composition to produce the coating on at least one surface of the sponge.

The invention is also a method of applying a cosmetic or medicine to skin, comprising applying the cosmetic or medicine to a coated natural or synthetic sponge of the invention and contacting the coated natural or synthetic sponge with applied cosmetic or medicine to the skin such that the cosmetic or medicine is transferred from the coated natural or synthetic sponge to the skin.

The sponge is a water-insoluble, flexible, cellular material that has an interconnected pore system open to at least the external surface or surfaces to which the coating is to be applied. The sponge may be a natural sponge such as a member of the phylum Porifera or a luffa such as Luffa aegyptiaca or Luffa acutangula. Alternatively, the sponge may be non-natural cellular material that is made from a polymeric material such as cellulose, a polyurethane rubber, a natural rubber, a silicone polymer, a homopolymer or copolymer of a conjugated diene (such as a butadiene and/or isoprene homopolymer or copolymer, including butadiene-styrene polymers and isoprene-styrene copolymers), polychloroprene, a homopolymer or copolymer of one or more acrylate monomers such as methyl acrylate, ethyl acrylate, hydroxyethylacryate, butyl acrylate and the like; a homopolymer and/or copolymer of isobutylene; a nitrile rubber; a polysulfide rubber, a silicone rubber; a homopolymer or and copolymer of neoprene, and the like. The polymeric material may have a glass transition temperature of 0° C. or lower as measured by differential scanning calorimetry.

The sponge (without the coating) in some embodiments exhibits a moisture wicking time of 5 seconds or less, preferably 4 seconds or less at 23 ± 2° C. Moisture wicking time is measured on 5.08 X 5.08 X 2.54 cm skinless samples that are dried to constant weight. 3 mL of room temperature water is slowly dropped onto the top surface of the sponge sample from a pipette and the amount of time required for the sponge to absorb the water is recorded as the wicking time.

The sponge (without the coating) may have, for example, a foam density of at least 24 kg/m³, at least 32 kg/m³, at least 36 kg/m³ or at least 40 kg/m³, as measured according to ASTM D-3574. The foam density may be, for example, up to 250 kg/m³, up to 200 kg/m³ or up to 175 kg/m³.

The sponge (without the coating) may exhibit an elongation to break of at least 50%, at least 75% or at least 100%.

The sponge (without the coating) may exhibit a compression force deflection (CFD) value of 0.4 to 15.0 kPa, and more preferably 0.4 to 10 kPa, 0.4 to 5 kPa, 0.4 to 2.5 kPa or 0.4 to 1.5 kPa, at 40% compression, as measured according to ISO3386-1.

The sponge (without the coating) may exhibit a resiliency of up to 70%, up to 60%, up to 50%, up to 25%, up to 20%, up to 15% or up to 10% on the ball rebound test of ASTM D-3574. In some embodiments the sponge exhibits a resiliency of 20% to 70%, 40% to 70%, or 50% to 70%.

The sponge (without the coating) in some embodiments exhibits a recovery time of at most one second or at most 0.25 second or at most 0.1 second. Recovery time for purposes of this invention is measured by compressing a 2.0-inch (5.08 cm) thick piece of the sponge (4.0 x 4.0 x 2.0 inches, 10.16 x 10.16 x 5.08 cm) to 24% of its original thickness at room temperature, holding the sponge at that compression for one minute and releasing the compressive force. The time required after the compressive force is released for the sponge to regain 90% of original thickness is the recovery time. Recovery time is conveniently measured using a viscoelastic foam-testing device such as a RESIMAT 150 device (with factory software) from Format Messtechnik GmbH. In alternative embodiments, the sponge (without the coating) exhibits a recovery time of at least one second or at least 2 seconds and up to 15 seconds, preferably up to 10 seconds.

The sponge (without the coating) may exhibit an airflow of at least 0.8 L/s as measured according to ASTM D3574 test G. The airflow may be at least 1.2 L/s or at least 1.4 L/s and may be, for example, up to 8 L/s, up to 6 L/s or up to 4 L/s.

Polyurethane foams having the foregoing characteristics and which are useful as the sponge can be prepared using general methods such as are described in, for example, in WO 2017/210439, U.S. Pat. Nos. 4,365,025, 6,479,433, 8,809,410, 9,814,187 and 9,840,575, U.S. Published Pat. Application Nos. 2004-0049980, 2006-0142529 and 2016-0115387, and PCT/US2018/052323, among many others.

The sponge is generally of small dimensions, having a volume of, for example, 200 cm³ or less and often no greater than 100 cm³ or no greater than 50 cm³. Its thickness (smallest orthogonal dimension) is in some embodiments no greater than 3.81 cm, no greater than 2.54 cm or no greater than 1.27 cm. The sponge may be molded, i.e., prepared in a mold in which the internal geometry is the same as the external geometry of the sponge. The sponge may be a cut sponge made by fabricating a larger sponge body to the final dimensions and geometry of the sponge.

The cured coating includes an elastomeric, non-silicone polymer that is a room temperature (23° C.) solid and insoluble in water. The elastomeric, non-silicone polymer by itself (i.e., in the absence of the phase change material and ceramic particles) preferably has a glass transition temperature of no greater than 0° C. as measured by differential scanning calorimetry and an elongation to break of at least 50% per ASTM D412. A polymer having those characteristics is considered for purposes of this invention to be elastomeric. The elastomeric, non-silicone polymer by itself may have a glass transition temperature of no greater than -15° C., no greater than -25° C. or no greater than -40° C. Its elongation to break may be 100% or more.

Examples of suitable elastomeric, non-silicone polymers include natural rubber and synthetic polymers such as homopolymers and copolymers of conjugated dienes such as butadiene and isoprene (such as styrene-butadiene rubbers); homopolymers and copolymers of acrylate monomers such as methyl acrylate, ethyl acrylate, hydroxyethylacryate, butyl acrylate and the like; homopolymers and copolymers of isobutylene; nitrile rubbers; polysulfide rubbers; homopolymers and copolymers of neoprene; polyurethane rubber and the like. Preferred elastomeric, non-silicone polymers include acrylate polymers, i.e., homopolymers and copolymers of one or more acrylate monomers such as methyl acrylate, ethyl acrylate, hydroxyethylacrylate, butyl acrylate and the like. Natural and/or synthetic latex foams are useful.

The elastomeric, non-silicone polymer(s) preferably constitutes at least 15 weight percent, at least 20 weight percent or at least 30 weight percent of the combined weight of the non-silicone polymer, silicone polymer, encapsulated phase change material particles and ceramic particles. In some embodiments, the elastomeric, non-silicone polymer constitutes up to 75 weight percent, up to 50 weight percent or up to 40 weight percent, on the same basis.

The coating contains (i) at least one silicone polymer having a kinematic viscosity of at least 1 million mm²/s at 25° C. and/or a Williams plasticity number of at least 30 as measured according to ASTM 926; (ii) particles of an encapsulated phase change material and optionally (iii) ceramic particles having a particle size of up to 50 µm. The encapsulated phase change material and ceramic particles (when present) in some embodiments are embedded in the elastomeric, non-silicone polymer.

The silicone polymer is characterized in having a polymer chain of alternating silicon and oxygen atoms. The silicon atoms are also bonded to organic groups. The silicone polymer has a sufficiently high molecular weight to provide a kinematic viscosity greater of at least 1 million mm²/s at 25° C. as measured by rotational shear rheometry. A suitable measurement device is a TA Dynamic Hybrid Rheometer or Anton Paar Modular Compact Rheometer equipped with a 25 mm diameter cone and plate geometry. The kinematic viscosity may be at least 5 million mm²/s, at least 10 million mm²/s or at least 25 million mm²/s and may be, for example, up to 500 million mm²/s, up to 250 million mm²/s or up to 150 million mm²/s.

The organic groups bonded to the silicon atoms of the silicone polymer may be independently selected from hydrocarbon or halogenated hydrocarbon groups. These may be specifically exemplified by alkyl groups having 1 to 20 carbon atoms, such as methyl, ethyl, propyl, butyl, pentyl and hexyl; cycloalkyl groups, such as cyclohexyl and cycloheptyl; aryl groups having 6 to 12 carbon atoms, such as phenyl, tolyl and xylyl; aralkyl groups having 7 to 20 carbon atoms, such as benzyl and phenylethyl; and halogenated alkyl groups having 1 to 20 carbon atoms, such as 3,3,3-trifluoropropyl and chloromethyl. The silicone polymer can be a homopolymer, a copolymer or a terpolymer containing such organic groups. Examples include homopolymers comprising dimethylsiloxy units, homopolymers comprising 3,3,3-trifluoropropylmethylsiloxy units, copolymers comprising dimethylsiloxy units and phenylmethylsiloxy units, copolymers comprising dimethylsiloxy units and 3,3,3-trifluoropropylmethylsiloxy units, copolymers of dimethylsiloxy units and diphenylsiloxy units and interpolymers of dimethylsiloxy units, diphenylsiloxy units and phenylmethylsiloxy units, among others.

The silicon-bonded organic groups of the silicone polymer may also be selected from alkenyl groups having 1 to 20 carbon atoms, such as vinyl, allyl, butyl, pentyl, hexenyl, or dodecenyl. Examples include dimethylvinylsiloxy-endblocked dimethylpolysiloxanes, dimethylvinylsiloxy-endblocked dimethylsiloxane-methylvinylsiloxane copolymers, dimethylvinylsiloxy-endblocked methylphenylpolysiloxanes; dimethylvinylsiloxy-endblocked methylphenylsiloxane-dimethylsiloxane-methylvinylsiloxane copolymers.

The silicon-bonded organic groups of the silicone polymer may also be selected from various organofunctional groups such as amino, amido, mercapto, or epoxy functional groups.

The silicone polymer may be linear, branched, or a mixture of linear and branched species.

Two or more silicone polymers may be used in combination.

In some embodiments, the silicone polymer includes at least one hydroxyl-terminated silicone polymer that has a kinematic viscosity of at least 10 million mm²/s and preferably 20 million mm²/s to 150 million mm²/s. Such hydroxyl-terminated silicone polymer is preferably a poly(dimethyl siloxane).

In some embodiments, the silicone polymer includes at least one vinyl-terminated silicone polymer that has a kinematic viscosity of at least 10 million mm²/s and preferably 20 to 200 million mm²/s. Such vinyl-terminated silicone polymer is preferably a poly(dimethylsiloxane). A specific example is a divinylmethicone/dimethicone copolymer.

In some embodiments, the silicone polymer includes (a) at least one poly(dimethylsiloxane) having a kinematic viscosity of 10 million mm²/s to 75 million mm²/s and (b) at least one poly(dimethylsiloxane) having a kinematic viscosity of greater than 75 million mm²/s and up to 200 million mm²/s, preferably up to 150 million mm²/s. In such embodiments, silicone polymers (a) and (b) may both be hydroxyl-terminated; both may be vinyl-terminated, and one may be hydroxyl terminated and one may be vinyl-terminated. In particular embodiments, the silicone polymer is a mixture of a hydroxyl-terminated poly(dimethylsiloxane) having a kinematic viscosity of 10 million mm²/s to 75 million mm²/s and a vinyl-terminated poly(dimethylsiloxane) having a kinematic viscosity of greater than 75 million mm²/s and up to 200 million mm²/s, preferably up to 150 million mm²/s.

The silicone polymer(s) constitute 2.5 to 30 weight percent of the combined weight of the elastomeric, non-silicone polymer, silicone polymer, encapsulated phase change material particles and ceramic particles (when present). In some embodiments, the silicone polymer constitutes at least 7.5 weight percent or at least 10 weight percent, on the same basis, and in some embodiments may constitute up to 25 weight percent or up to 20 weight percent, again on the same basis.

The encapsulated phase change material includes a phase change material that has a melting or glass transition temperature of 25° C. to 37° C., which phase change material is contained within a shell. The weight of the phase change material, for purposes of this invention, includes the weight of the shell. The shell may constitute, for example, 5 to 25% of the total weight of the encapsulated phase change material, the phase change material itself constituting the remainder thereof, i.e., 75 to 95% by weight thereof.

The phase change material may be or contain, for example, any one or more of a natural or synthetic wax such as a polyethylene wax, bees wax, lanolin, carnauba wax, candelilla wax, ouricury wax, sugarcane wax, jojoba wax, epicuticular wax, coconut wax, petroleum wax or paraffin wax. The phase change material in some embodiments is or includes an alkane having 14 to 30, especially 14 to 24 or 16 to 22 carbon atoms, or a mixture of any two or more of such alkanes. In a specific embodiment, the phase change material is or includes octadecane and/or eicosane. The phase change material preferably has a melting temperature of 25 to 37° C., especially 25 to 32° C. or 28 to 32° C.

The encapsulated phase change material may exhibit a heat of fusion within the temperature range of 25 to 37° C. of at least 50 Joule/gram (J/g), at least 100 J/g or at least 150 J/g, as measured by differential scanning calorimetry. The heat of fusion may be as much as 300 J/g or more, but is more commonly up to 250 J/g or up to 200 J/g.

The shell material may be, for example, a polymeric material that has a melting or decomposition (if the polymeric material decomposes without melting) temperature of at least 50° C. and preferably at least 100° C. Examples of useful shell materials include crosslinked thermoset resins such as crosslinked melamine-formaldehyde, crosslinked melamine, crosslinked resorcinol urea formaldehyde and gelatin.

The encapsulated phase change material is in the form of particles. The particles may have particle sizes of 100 nm to 100 µm as measured by microscopy. In some embodiments, the particles have particle sizes of at least 250 nm, at least 500 nm, at least 1 µm or at least 5 µm, and up to 75 µm or up to 50 µm.

Suitable methods for preparing the encapsulated phase change material are described, for example, in U.S. Pat. Nos. 10,221,323 and 10,005,059.

Suitable encapsulated phase change materials are available from Microtek Laboratories, Dayton, Ohio, US.

The encapsulated phase change material constitutes 10 to 70 weight percent of the combined weight of the elastomeric, non-silicone polymer, silicone polymer, encapsulated phase change material and ceramic particles (when present). In some embodiments the encapsulated phase change material constitutes at least 20 weight percent, at least 30 weight percent or at least 40 weight percent on the foregoing basis, and up to 60 weight percent or up to 50 weight percent, on the same basis.

The optional ceramic particles are generally characterized as being non-metallic inorganic solids at 23° C. and having a melting or decomposition (if the ceramic particles decompose without melting) temperature of at least 200° C. The ceramic material should be insoluble in water and other components of the coating composition used to coat the sponge. The ceramic material is a compound of at least two chemical elements, of which at least one is a non-metal. The ceramic particles may be amorphous, semicrystalline or crystalline, but do not undergo a phase change in the temperature range of 0 to 50° C. The ceramic material preferably has a thermal conductivity of at least 50 W/(m·K) in at least one direction, as measured according to ASTM C1470. Examples of useful ceramic particles include boron nitride, which may be amorphous or in the hexagonal, cubic and/or wurtzite form, and silicon nitride.

The ceramic particles have a particle size of up to 50 µm. Particle sizes herein refer to the longest dimension of primary (non-agglomerated) particles, as determined using microscopic methods. A preferred minimum particle size is at least 100 nm, at least 250 nm or at least 500 nm. A preferred maximum particle size is up to 20 µm, up to 10 µm or up to 5 µm.

The ceramic particles, when present, constitute up to 25 weight percent of the combined weight of the elastomeric non-silicone polymer, silicone polymer, encapsulated phase change material particles and ceramic particles. In some embodiments the ceramic particles constitute at least 2 weight percent or at least 4 weight percent on the same basis, and constitute up to 20 weight percent, up to 15 weight percent or up to 10 weight percent, again on the same basis.

The coating in some embodiments is produced by forming an emulsion and/or dispersion of the elastomeric non-silicone polymer, encapsulated phase change material and (optionally) the ceramic particles, applying the emulsion or dispersion to a surface of the sponge and curing the emulsion to produce the cured coating. “Curing” is used in this context to simply mean that the coating composition is formed into a solid coating by any mechanism or combination of mechanisms as appropriate for the particular elastomeric non-silicone polymer that is present. It is not necessary that any chemical reaction (such as, for example, polymerization, crosslinking or chain extension) take place during the curing step, although such a reaction may take place in some cases. Curing may simply involve drying the applied emulsion or dispersion to produce a solid coating.

A coating composition in the form of an emulsion or dispersion includes a continuous liquid phase. The continuous liquid phase contains water and/or one or more other compounds that are liquid at room temperature (23° C.) and having a boiling temperature at standard pressure of 40 to 100° C.; such materials may constitute, for example, 10 to 50% of the total weight of the coating composition. The elastomeric, non-silicone polymer is dispersed in the continuous liquid phase in the form of particles or droplets. The silicone polymer preferably is dispersed in the continuous liquid phase in the form of droplets. The particles of the encapsulated phase change material and the ceramic particles also are dispersed therein. The emulsion preferably is aqueous, i.e., the continuous liquid phase includes water. Preferably the emulsion or dispersion contains no more than 10% by weight, especially no more than 5% or no more than 2%, of room temperature liquid organic compounds that have a boiling temperature at standard pressure of 40 to 100° C., based on the combined weight of such organic compounds and water.

The elastomeric non-silicone polymer may be provided in the form of an emulsion produced in a emulsion polymerization process, in which one or more monomers are dissolved or dispersed into a liquid phase and subjected to polymerization conditions until the polymer chains precipitate and are converted to polymer particles dispersed in a liquid phase.

Similarly, an emulsion or dispersion of the elastomeric non-silicone polymer can be produced in a mechanical dispersion process in which molten elastomeric, non-silicone polymer is dispersed into a liquid phase and the cooled to solidify the elastomeric non-silicone polymer.

In yet another suitable process, the elastomeric non-silicone polymer may be ground or otherwise formed into small particles that are then dispersed in a liquid phase.

The liquid phase of any such emulsion or dispersion of the elastomeric, non-silicone polymer may form a portion of or all of the liquid phase of the coating composition.

The silicone polymer preferably is provided in the form of an emulsion in a liquid phase, preferably an aqueous liquid phase. Such an emulsion may contain, for example, 10 to 70 weight percent and especially 40 to 70 weight percent of the silicone polymer, the remainder of the emulsion including water and one or more surfactants. Examples of such silicone polymer emulsions are described, for example, in WO 2012/018750 and WO 2019/081277. The surfactant is preferably non-ionic. Ethylene oxide/propylene oxide block copolymers and ethoxylated fatty alcohols are examples of useful non-ionic surfactants for use in such a silicone polymer emulsion.

In such an emulsion of the silicone polymer, the silicone polymer advantageously is present in the form of droplets having a Dv50 of at most 20 µm. Dv50 refers to a droplet size at which 50 volume percent of the droplets have the same or smaller diameter. The Dv50 may be at most 10 µm in some embodiments. In some embodiments, at least 90 volume percent of the droplets have particle sizes from 100 nm to 10 µm.

The liquid phase of any such emulsion of the silicone polymer may form a portion of or all of the liquid phase of the coating composition.

A coating composition in the preferred form of an emulsion and/or dispersion is conveniently formed by combining an emulsion or dispersion of the elastomeric, non-silicone polymer with the silicone polymer, encapsulated phase change particles and the ceramic particles, at proportions as indicated before. The silicone polymer is preferably also provided in the form of an emulsion, as described before.

Such a coating composition may include one or more optional materials, in addition to those already described.

Among the useful optional materials are one or more hydrophilic polymers that are liquid at room temperature (23° C.) and have a weight average molecular weight of 350 to 8,000 g/mol, especially 350 to 1200 g/mol or 350 to 800 g/mol by gel permeation chromatography. The hydrophilic polymer preferably is water-soluble. Such a hydrophilic polymer may contain at least 50 weight-% or at least 75 weight-% oxyethylene units, and may be, for example a homopolymer of ethylene oxide or a copolymer (random and/or block) of ethylene oxide and one or other alkylene oxides such as 1,2-propylene oxide. Such a hydrophilic polymer, when present, may constitute 0.1 to 15 percent of the combined weight of the hydrophilic polymer, elastomeric non-silicone polymer, silicone polymer, phase change material and ceramic particles. A preferred amount is at least 1, at least 2, at least 4 or at least 5 weight-percent and up to 12, up to 10 or up to 8 weight percent, on the same basis.

Another useful optional material is one or more surfactants, which can perform one or more useful functions. Such a surfactant may function as a wetting agent, facilitating the dispersion of the particles of the phase change material and/or the ceramic particles into the remaining ingredients of the coating composition. A surfactant may function as a defoamer or deaerator, to reduce the entrainment of gases by the coating composition and reduce bubbles. Various silicone surfactants are useful for these purposes, as well as various non-silicone surfactants such as sulfate esters, sulfonate esters, phosphate esters, ethoxylates such as ethoxylated fatty alcohols, fatty acid esters, amine oxides, sulfoxides, ethylene oxide/propylene oxide block copolymers and phosphine oxides. A surfactant may be nonionic, anionic, cationic or zwitterionic. One or more surfactants may constitute, for example, 0.1 to 5 weight-percent of the total weight of the coating composition. A surfactant present in an emulsion of the silicone polymer may form all or a part of the surfactants in the coating composition.

Other useful ingredients include various rheology modifiers such as various thickeners and thixotropic agents. Among these are fumed silica and various water-soluble or water-swellable polymers of acrylic acid that contain free acid groups or carboxylic acid salt groups (such as, for example, alkali metal, ammonium (NH₄), quaternary ammonium, or quaternary phosphonium carboxylic acid salts). Particularly useful rheology modifiers include aqueous emulsions of crosslinked acrylic acid polymers, such as are sold by DuPont under the trade designation Acrysol®. Specific examples are Acrysol® ASE-60 and Acrysol ASE-95 emulsions. When present, such rheology modifiers may constitute, for example, 0.01 to 5 weight-percent, preferably 0.05 to 1 weight percent, of the coating composition.

Still other useful ingredients include one or more colorants, preservatives, antioxidants and biocides.

The coating composition is conveniently prepared by mixing the foregoing ingredients. When the elastomeric, non-silicone polymer is provided in the form of an emulsion or dispersion, it is convenient to combine the remaining ingredients with the emulsion or dispersion of the elastomeric non-silicone polymer in any convenient order to produce a homogeneous dispersion.

A useful way of producing a coating composition of the invention is to charge a portion of the liquid phase to a vessel. The hydrophilic polymer, if used, is mixed with this portion of the liquid phase, in the absence of the elastomeric, non-silicone polymer and silicone polymer. The ceramic particles (when used) are then combined with the portion of the liquid phase (and hydrophilic polymer, if used) in the vessel, followed by adding the elastomeric, non-silicone polymer, preferably in the form of an emulsion or dispersion, the silicone polymer (again preferably in the form of an emulsion), the encapsulated phase change material and other ingredients in any convenient order.

The coating composition can be applied to at least one external surface of the sponge. The coating method is not particularly critical. Rolling, brushing, spraying, immersion or other coating methods are suitable.

Enough of the coating composition preferably is applied that, after curing, a cured coating having a thickness of 100 µm to 10 mm is produced on at least one surface of the sponge. The coating thickness is preferably at least 250 µm or at least 350 µm and up to 2,500 µm, up to 1500 µm or up to 1000 µm. The coating, after drying, may constitute, for example, 1 to 25% of the combined weight of the coating and sponge.

The coating on an external surface of the sponge may be continuous but preferably is discontinuous such that at least some of the pores within the sponge remain open at the coated external surface.

Internal surfaces of the sponge may or may not be coated. In some embodiments, no more than 25% or no more than 10% of the interior surfaces of the sponge are uncoated. In other embodiments, greater than 25% or greater than 50%, and as much as 100%, of the interior surfaces of the sponge are coated. As before, the coating preferably is discontinuous such that pores within the sponge remain open at the coated exterior surfaces.

The coating composition is cured on the surface of the sponge. The curing method may depend somewhat on the particular elastomeric non-silicone polymer and/or on the physical form of the coating composition. The curing of a coating composition in the form of an emulsion includes at least a drying step of removing water and/or one or more other compounds that are liquid at room temperature (23° C.) and having a boiling temperature at standard pressure of 40 to 100° C., as may be present in the coating composition. Such a drying step can be performed at approximately room temperature, such as from 15 to 30° C., or at an elevated temperature such as great than 30° C. up to 100° C. or more.

If curing includes a chemical reaction (such as, for example, polymerization, crosslinking or chain extension), conditions of the curing reaction, such as temperature, the presence of coreactants, catalysts, initiators, etc. not otherwise present in the coating composition, etc., are selected to facilitate the chemical reaction to complete the cure.

The coated sponge in some embodiments exhibits a sliding resistance value (on a coated surface) of at most 50, at most 40 and more preferably 20 to 40 or 30 to 40; a thermal cooling value of at least 4, preferably at least 5 and especially 5 to 10; and/or a thermal persistence value of at least 3, at least 4 or at least 5 and preferably 5 to 10, all as measured using the BioTac® Toccare apparatus (Syntouch BioTac® Product Manual, V. 21, August 2018) at a temperature of 24 to 25° C. and a relative humidity of 40 to 50%. The coated sponge in some embodiments exhibits a durometer harness of at most 15 on the 00 scale as measured according to ASTM D2240. The presence of the coating in general has been found to decrease sliding resistance and increase both thermal cooling and thermal persistence, compared to the uncoated sponge.

In addition, in some embodiments the coated sponge exhibits an adhesive tack value of less than 5, as measured in the same manner using the BioTac® Toccare apparatus. The presence of the coating is generally found to decrease this value, compared to that of the uncoated sponge.

The coated sponge is useful for applying cosmetic and/or medicinal formulations to the skin. The cosmetic and/or medicinal formulation may be, for example, a low viscosity liquid, a lotion, a cream, a gel, a paste or a powder. All that is necessary that the formulation be one that is capable of being sorbed onto and/or otherwise adhered to the coated sponge and then transferred from the sponge to the skin. Examples of such cosmetic and/or medicinal formulations include antiperspirants and deodorant; skin care creams; skin care lotions; moisturizers; facial treatments such as acne or wrinkle removers; personal and facial cleansers; bath oils; perfumes; colognes; sachets; sunscreens; pre-shave and after-shave lotions; shaving soaps and shaving lathers; hair shampoos; hair conditioners; hair colorants; hair relaxants; hair sprays; mousses; gels; permanents; depilatories; cuticle coats; make-ups; color cosmetics; foundations; concealers; blushes; lipsticks; eyeliners; mascara; oil removers; color cosmetic removers; powders; medicament creams; pastes or sprays including antiacne, dental hygienic, antibiotic, healing promotives and nutritive, and the like.

The coated sponge can be used in conventional manner to apply a cosmetic and/or medicinal formulation. The cosmetic and/or medicinal formulation can be applied to the coated sponge in any convenient manner as may be suitable for the product form of the formulation. Dipping, immersion, rubbing, brushing, wiping and other methods can be used. The coated sponge with the applied cosmetic and/or medicinal formulation is then contacted with the skin to transfer the cosmetic and/or medicinal formulation onto the skin. Again, the manner of contact is not particularly critical and will in general be adapted to the nature of the cosmetic and/or medicinal formulation as well as the area of the skin to which it is to be applied.

Similarly, the coated sponge can be used to clean the skin in the same manner as the uncoated sponge. Soil, foreign objects, cosmetics such as make-up, and the like can be removed, among many other things. The haptic properties of the coated sponge are quite advantageous for this use.

The following examples are provided to illustrate the invention, but are not intended to limit the scope thereof. All parts and percentages are by weight unless otherwise indicated.

The Deaerator is a polyether siloxane copolymer with fumed silica, sold as Tego Airex 904W by Evonik.

PEG is a polyethylene glycol having an average nominal hydroxyl functionality of 2 and a number average molecular weight of approximately 600 g/mole.

Silicone Emulsion A is an aqueous emulsion containing a hydroxyl-terminated polydimethylsiloxane, ethoxylated C11-15 secondary alcohols and glycol-modified trimethylated silica. It is sold commercially by The Dow Chemical Company as Dowsil™ 52. The hydroxyl-terminated polydimethylsiloxane by itself has a dynamic viscosity of 30 million mm²/s at 0.01 Hz and 25° C. as measured by rotational shear rheometry using a TA Dynamic Hybrid Rheometer or Anton Paar Modular Compact Rheometer equipped with a 25 mm diameter cone and plate geometry. The hydroxyl-terminated polydimethylsiloxane is present in the emulsion in the form of spherical droplets having a Dv50 equal to or less than 10 microns, as measured by laser diffraction with a fixed scattering angle of 90° or 180°. The emulsion contains about 65% by weight of the polydimethylsiloxane.

Acrylic Emulsion A is an acrylic latex polymer emulsion with 55% solids by weight. The latex particles are an elastomeric polymer having a T_{g} of approximately -50° C. It is available as Rhoplex® E-1791 from The Dow Chemical Company.

Acrylic Emulsion B is an acrylic latex polymer emulsion with 55% solids by weight. The latex particles are an elastomeric polymer having a T_{g} of approximately -50° C. It is available as Rhoplex® 3166 from The Dow Chemical Company.

PCM 1 is a microencapsulated paraffin wax having a particle size of 15 to 30 µm. The wax constitutes 85-90% of the weight of the material, a polymeric shell constituting the remainder of the weight of the product. The wax has a melting temperature of approximately 32° C. The product has an enthalpy of melting of 160-170 J/g. It is commercially available as MPCM 32D from Microtek Laboratories.

PCM 2 is a microencapsulated paraffin wax having a particle size of 15 to 30 µm. The wax constitutes 85-90% of the weight of the material, a polymeric shell constituting the remainder of the weight of the product. The wax has a melting temperature of approximately 28° C. The product has an enthalpy of melting of 180-190 J/g. It is commercially available as MPCM 28D from Microtek Laboratories.

PCM 3 is a 70% solids slurry of PCM 1 in a diluent.

RM (rheology modifier) 1 is an aqueous emulsion containing cross-linked acrylate particles having acid groups. The solids content is 28%. When diluted with water and neutralized with a base (NH₄OH), this product acts as a thickener.

RM 2 is an aqueous emulsion containing cross-linked acrylate particles having acid groups. The solids content is 18%. When diluted with water and neutralized with a base (NH₄OH), this product acts as a thickener.

NH₄OH is a 28-30% ammonium hydroxide solution, for neutralizing RM 1 and/or RM 2.

BN is boron nitride (at least 98% pure), in the form of platelets having a longest dimension of about 1 to 3 µm, available from Wego Chemical.

Silicone Emulsion B is a nonionic emulsion containing 60% of a divinyldimethicone/dimethicone copolymer that has terminal vinyl groups, an ethoxylated mixture of C₁₁₋₁₅ linear alkanols having about 23 ethoxy groups per molecule and an ethyoxylated mixture of C₁₁₋₁₅ linear alkanols having about 3 ethoxy groups per molecule. The divinyldimethicone/dimethicone copolymer by itself has a dynamic viscosity of at least 120 million mm²/s at 0.01 Hz and 25° C. as measured by rotational shear rheometry using a TA Dynamic Hybrid Rheometer or Anton Paar Modular Compact Rheometer equipped with a 25 mm diameter cone and plate geometry, and is present in the emulsion in the form of spherical droplets having a Dv50 particle size of 0.6 µm, as measured by laser diffraction with a fixed scattering angle of 90 ° or 180 °. This product is sold commercially as Dowsil™ HMW 2220 Non-Ionic Additive.

Coating compositions 1 and 2 are made from the ingredients listed in Table 1 by mixing them in a high-speed laboratory mixer to produce a homogeneous mixture.

TABLE 1

| Ingredient | Parts by weight | |
|---|---|---|
| | Coating 1 | Coating 2 |
| Water | 23.8 | 29.0 |
| Deaerator | 0.3 | 0.1 |
| PEG | 6.1 | 7.8 |
| Silicone Emulsion A | 2.3 | 2.6 |
| Acrylic Emulsion A | 42.1 | 0 |
| Acrylic Emulsion B | 0 | 29.0 |
| PCM 1 | 0 | 1.1 |
| PCM 2 | 21.5 | 18.3 |
| PCM 3 | 1.5 | 0 |
| RM 1 | 0.3 | 0.3 |
| RM 2 | 1.0 | 0.1 |
| NH₄OH | 0.6 | 0.5 |
| BN | 0 | 2.6 |
| Silicone Emulsion B | 0 | 8.6 |
| Approximate PCM Content, Wt.-%¹ | 47.6% | 43.2% |
| Approximate Silicone Content, Wt.-%¹ | 3.4% | 15.4% |
| Approximate BN content, Wt.-%¹ | 0% | 5.8% |

¹ Based on the combined weight of the elastomeric polymer, the PCM, the BN and the silicones.

The coating compositions each are used to produce a coating on a commercially available polyurethane cosmetic sponge. The sponge is sold as Aesthetica Body Sponge that, prior to coating, has flat sides and is oval in shape. It weighs approximately 13.75 grams, has a thickness of approximately 2 cm and has a foam density of about 0.17 g/cm³. Its volume is approximately 81 cm³. The sponge is resilient and springs back rapidly when compressed. It has open cells and lacks a surface skin.

In each case, 2.75 to 3 grams of the coating composition are applied to one flat side of the sponge and spread evenly across the surface using a flat blade. The applied coating is cured by heating the coated sponge at 80° C. for 60 minutes. Coating 1, being more viscous, blocks off many of the surface pores. Coating 2 does not block off pores on the coated surface.

Sliding resistance, thermal cooling and thermal persistence of the coated surface and the opposing uncoated surface are evaluated using a BioTac® Toccare device (Suntouch, Montrose, CA), which reports values for each attribute on a relative scale. Testing is performed at a temperature of 24-25° C. and a relative humidity of 47-48%. Measurements are taken at five points on the mid-line of the sample (the line dividing the tested flat surface of the sample along the length of the longest dimension). For the intended cosmetic applications, a lower value for sliding resistance is preferred, and higher values are preferred for thermal cooling and thermal persistence. Results are as indicated in Table 2.

TABLE 2

| Test | Coating 1 | | Coating 2 | |
|---|---|---|---|---|
| | Uncoated Side | Coated Side | Uncoated Side | Coated Side |
| Sliding Resistance | 48.1 | 37.3 | 52.2 | 36.6 |
| Thermal Cooling | 3.5 | 6.9 | 3.5 | 5.8 |
| Thermal Persistence | 2.7 | 7.2 | 2.7 | 6.0 |
| Adhesive Tack | 6.8 | 3.9 | 5.1 | 4.9 |

Significant improvements in all three of these criteria are obtained by applying the coating to the sponge surface. Sliding resistance value drops by 10 units or more. Changes of this magnitude are easily perceptible by human users.

The increases in thermal cooling and thermal persistence values are indicative of a greater wanted “cooling” sensation as well as a longer duration of that sensation. The magnitudes of these increases are such that they are perceptible by a human user.

Cylindrical sponge samples 2.54 cm in diameter, 2 cm in length and weighing 1.78 ± 0.02 g are punched from an uncoated sample of the same cosmetic sponge. The sponge samples are immersed in one or the other of the coating compositions described in Table 1, then hung in an 80° C. oven for 60 minutes to dry. The weight of the coated and dried sponge samples is 2.02 ÷ 0.15 g, corresponding to about 12% by weight coating. The coating covers substantially all exterior surfaces of the sponge, with little penetration into the interior surfaces. The pores of the sponge remain open and interconnected.

The coated sponge samples are then submerged in a liquid make-up (L’Oreal True Match™ Nude Beige Super-Blendable Makeup Titanium Dioxide Sunscreen) for 5 seconds, removed, and excess liquid make-up is allowed to drip off the sponge sample for 5 seconds. Each sample is then weighed to determine the amount of make-up absorbed. The samples are then compressed by 25% (to 1.5 cm thickness) for 5 seconds to expel make-up using a hand-actuated plunger equipped with a pressure distribution plate, and re-weighed to determine the amount of make-up released. Uncoated sponge samples are evaluated the same way for comparison. Results are as indicated in Table 3.

TABLE 3

| Sample | Coating 1 | | | Coating 2 | | |
|---|---|---|---|---|---|---|
| | Make-up Absorbed (g) | Make-up Released (g) | % Make-Up Released | Make-up Absorbed (g) | Make-up Released (g) | % Make-Up Released |
| Uncoated | 4.98 | 1.64 | 32.9 | 4.98 | 1.64 | 32.9 |
| Coated | 3.38 | 1.82 | 54.1 | 3.57 | 1.94 | 54.4 |

The coated sponges are seen to be dramatically more efficient in transferring make-up. Whereas the uncoated sponges absorb more make-up, they transfer less, retaining about two-thirds of the absorbed make-up. The coated sponges absorb less make-up but transfer more, releasing over half of the absorbed make-up. The coated sponges apply as much make-up as the uncoated sponge while consuming approximately one-third less of the cosmetic product, thereby greatly decreasing the amount of wasted product.

1. An article comprising a natural or synthetic sponge and a cured coating of a solid, water-insoluble, elastomeric non-silicone polymer adhered to at least one surface of the sponge, the cured coating further containing (i) at least one silicone polymer having a kinematic viscosity of at least 1 million mm²/s at 25° C. and/or a Williams plasticity number of at least 30 as measured according to ASTM 926; (ii) particles of an encapsulated phase change material, the phase change material having a melting or glass transition temperature of 25 to 37° C. and optionally (iii) ceramic particles having a particle size of up to 50 µm, the silicone polymer constituting 2.5 to 30 weight percent of the combined weight of the non-silicone polymer, silicone polymer, encapsulated phase change material particles and ceramic particles, the encapsulated phase change material constituting 10 to 70 weight percent of the combined weight of the non-silicone polymer, silicone polymer, encapsulated phase change material particles and ceramic particles, and the ceramic particles when present constituting up to 25 weight percent of the combined weight of the non-silicone polymer, silicone polymer, encapsulated phase change material particles and ceramic particles.

2. The article of claim 1 wherein the cured coating has a thickness of 100 to 2500 µm.

3. The article of claim 2 wherein the phase change material comprises any one or more of a natural or synthetic wax such as a polyethylene wax, bees wax, lanolin, carnauba wax, candelilla wax, ouricury wax, sugarcane wax, jojoba wax, epicuticular wax, coconut wax, petroleum wax or paraffin wax.

4. The article of claim 2 wherein the silicone polymer includes (a) at least one poly(dimethylsiloxane) having a kinematic viscosity of 10 million mm²/s to 75 million mm²/s, (b) at least one poly(dimethylsiloxane) having a kinematic viscosity of greater than 75 million mm²/s and up to 200 million mm²/s, preferably up to 150 million mm²/s or a mixture of (a) and (b).

5. The article of claim 2 wherein the silicone polymer is a mixture of a hydroxyl-terminated poly(dimethylsiloxane) having a kinematic viscosity of 10 million mm²/s to 75 million mm²/s and a vinyl-terminated poly(dimethylsiloxane) having a kinematic viscosity of greater than 75 million mm²/s and up to 200 million mm²/s,.

6. The article of claim 2 wherein the ceramic particles are present and the ceramic particles are boron nitride or silicon nitride particles having a particle size of 100 to 3000 µm.

7. The article of claim 2 wherein the phase change material constitutes 30 to 50 percent of the total weight of the elastomeric non-silicone polymer, silicone polymer, encapsulated phase change material particles and ceramic particles.

8. The article of claim 2 wherein the ceramic particles constitute 4 to 15 percent of the total weight of the elastomeric non-silicone polymer, silicone polymer, encapsulated phase change material particles and ceramic particles.

9. The article of claim 2 wherein the silicone polymer constitutes 10 to 20 percent of the total weight of the elastomeric non-silicone polymer, silicone polymer, encapsulated phase change material particles and ceramic particles.

10. The article of claim 2 wherein the cured coating further contains a hydrophilic polymer that is a liquid at 23° C. and has a weight average molecular weight of 350 to 8000, wherein the hydrophilic polymer constitutes 0.1 to 15 percent of the total weight of the elastomeric polymer, encapsulated phase change material particles, ceramic particles and hydrophilic polymer.

11. A coating composition comprising a liquid phase containing water and/or one or more other compounds that are liquid at 23° C. and have a boiling temperature at standard pressure of 40 to 100° C., a water-insoluble elastomeric, non-silicone polymer dispersed in the liquid phase in the form of particles or droplets, at least one silicone polymer having a viscosity of at least 1 million mm²/s at 25° C. and/or a Williams plasticity number of at least 30 as measured according to ASTM 926 dispersed in the liquid phase, particles of an encapsulated phase change material dispersed in the liquid phase, and ceramic particles dispersed in the liquid phase, wherein the silicone polymer constitutes 5 to 30 weight percent of the combined weight of the non-silicone polymer, silicone polymer, encapsulated phase change material particles and optionally ceramic particles, the encapsulated phase change material constitutes 10 to 70 weight percent of the combined weight of the non-silicone polymer, silicone polymer, encapsulated phase change material particles and ceramic particles, and the ceramic particles when present constitute up to 25 weight percent of the combined weight of the non-silicone polymer, silicone polymer, encapsulated phase change material particles and ceramic particles.

12. The coating composition of claim 11 wherein the phase change material comprises any one or more of a polyethylene wax, bees wax, lanolin, carnauba wax, candelilla wax, ouricury wax, sugarcane wax, jojoba wax, epicuticular wax, coconut wax, petroleum wax or paraffin wax, the ceramic particles are present and the ceramic particles are boron nitride or silicon nitride particles having a particle size of 100 to 3000 µm.

13. The coating composition of claim 12 further comprising a hydrophilic polymer that is a liquid at 23° C. and has a weight average molecular weight of 350 to 8000, wherein the hydrophilic polymer constitutes 0.1 to 15 percent of the total weight of the elastomeric polymer, encapsulated phase change material particles, ceramic particles and hydrophilic polymer.

14. A method of making a coated sponge, the method comprising applying the coating composition of claim 11 to at least one surface of a natural or synthetic sponge to form a film thereupon, and curing the coating composition to produce the coating on at least one surface of the sponge.

15. A method of applying a cosmetic or medicine to skin, comprising applying the cosmetic or medicine to a coated natural or synthetic sponge of claim 1 and contacting the coated natural or synthetic sponges with applied cosmetic or medicine to the skin such that the cosmetic or medicine is transferred from the coated natural or synthetic sponge to the skin.
